(19) United States
(12) Patent Application Publication
(10) Pub. No.: US 2010/0029545 A1
(43) Pub. Date: Feb. 4, 2010

(54) BORONIC ACID-CONTAINING BLOCK COPOLYMERS FOR CONTROLLED DRUG DELIVERY

(76) Inventors: Brent S. Sumerlin, Dallas, TX (US); Jennifer N. Cambre, Dallas, TX (US); Debashish Roy, Dallas, TX (US)

Correspondence Address: BAKER BOTTS L.L.P., 2001 ROSS AVENUE, SUITE 600, DALLAS, TX, 75201-2980, US

(21) Appl. No.: 12/534,916

(22) Filed: Aug. 4, 2009

Related U.S. Application Data
(60) Provisional application No. 61/086,064, filed on Aug. 4, 2008.

(51) Int. Cl. A61K 38/28; A61K 47/30; A61K 9/14

(52) U.S. Cl. 514/3; 514/772.3; 514/772.1; 528/8

(57) ABSTRACT

Polymeric nanoaggregate drug-delivery compositions including boron-containing copolymers are described. The drug-delivery compositions may further include a therapeutic agent comprising a nucleic acid, a polynucletide, a peptide, a polypeptide, a protein, a pharmaceutical or any combinations thereof. Methods for making the polymeric nanoaggregate compositions including at least one therapeutic agent (for example, insulin) as well as methods for administration of these compositions to mammals are also set forth. The disclosure also describes compositions and methods for controlled drug delivery. Further, polymeric nanoaggregate boron-containing compositions including insulin and methods for monitoring and regulating blood glucose levels of a mammal are also described. The disclosure also describes methods for treatment and/or control of diabetes mellitus by administering polymeric nanoaggregate boron-containing compositions including insulin to a mammal in need.

### PRIORITY CLAIM

The present application claims priority under 35 U.S.C. §119 to U.S. Provisional Patent Application No. 61/086,064, filed Aug. 4, 2008, which is incorporated by reference herein.

### FIELD OF THE DISCLOSURE

The present disclosure relates to compositions (e.g., nanoscale, polymer-based compositions comprising boron) and methods of making and using such compositions. For example, some embodiments of the disclosure relate to methods of making compositions and/or methods of administering compositions to a subject (e.g., a mammal).

### BACKGROUND OF THE DISCLOSURE

Diabetes affects about 170 million people worldwide and about 21 million Americans. Type I diabetes is caused by an insufficiency of insulin leading to elevated glucose levels in the bloodstream. Typical treatment regimens involve regular monitoring of blood glucose levels and frequent injections of insulin (e.g. by Insulin Pen or Insulin Pump). According to the National Institutes of Health, nearly 10% of the about 21 million Americans afflicted by diabetes are treated by traditional insulin injections. Complications of diabetes include heart disease and stroke, high blood pressure, blindness, kidney disease, amputations, and many others. Many of these complications can potentially be prevented by appropriate treatment, however, a strict regimen of constant glucose monitoring and painful/frequent insulin injections often leads to significantly reduced patient compliance with the prescribed treatment.

### SUMMARY

Therefore, a need has arisen for methods for monitoring glucose levels as well as for delivering insulin to a patient in need. Indeed, a need has arisen more broadly for methods and compositions for delivering a macromolecule (e.g., protein, peptide, and/or nucleic acid) to a subject (e.g., as a therapeutic agent). Therapeutic peptides, polypeptides or proteins (for example, hormones, growth factors) or specific genes to replace or supplement absent or defective genes are examples of therapeutics which may require such delivery systems.

According to some embodiments, one or more therapeutic agents (e.g., insulin), may be encapsulated in a polymer nanoaggregate of the disclosure. The disclosure also relates to methods of administering therapeutic compositions of the disclosure to a mammal in need thereof. In some embodiments, the disclosure relates to methods for preventing a disease or condition, treating a disease or condition, and/or reducing the symptoms of a disease or condition comprising administering a composition of the disclosure to a mammal in need thereof.

In some embodiments, a composition (e.g., a therapeutic composition) may include a polymer having a monomer unit comprising at least one boronic acid moiety. A boronic acid moiety may be attached to a monomer unit pendantly or terminally, according to some embodiments. A composition may include, in some embodiments, a block copolymer having one or more monomer units, wherein each monomer unit comprises at least one pendant boronic acid moiety. In some embodiments, a method of preparing a composition including a polymer having a monomer unit comprising at least one boronic acid moiety may include removing boronic or boronate pinacol esters from boron-containing polymers or copolymers by treatment with a boronic acid-functionalized resin.

In some embodiments, a composition (e.g., a therapeutic composition) may include a micelle and/or vesicle. A micelle and/or vesicle may have at least one boronic acid moiety (e.g., a block copolymer comprising monomer units having at least one pendant boronic acid moiety per repeat unit). A micelle and/or vesicle may include (e.g., enclose) one or more macromolecules according to some embodiments. A method for delivering a macromolecule (e.g., a therapeutic agent) may include, in some embodiments, dissolution of a micelle and/or vesicle (e.g., a micelle and/or vesicle comprising a block copolymer). According to some embodiments, a method for delivering a macromolecule may include dissolution of a micelle and/or vesicle in response to a change in concentration (e.g., local and/or global concentration) of a diol (e.g., 1,2-diol and/or a 1,3-diol). A method for delivering a macromolecule may include dissolution of a micelle and/or vesicle in response to an increase in concentration (e.g., local and/or global concentration) of a 1,2-diol or a 1,3-diol, in some embodiments. A method for delivering a macromolecule may include, in some embodiments, dissolution of a micelle and/or vesicle in response to an increase in concentration (e.g., local and/or global concentration) of a saccharide. Examples of a saccharide may include, in some embodiments, glucose, fructose, and/or sucrose. In some embodiments, a method for delivering a macromolecule (e.g., insulin) to a subject may include administering (e.g., orally administering and/or intravenous administration) a composition having a boronic acid-containing block copolymer.

In some embodiments, a composition including: a polymer having at least one monomeric boron moiety wherein the boron moiety may be incorporated pendantly or terminally; and a therapeutic agent are described. In some embodiments, the boron moiety may be a boronic acid. In some embodiments, the boron moiety may be a boronic ester.

In some embodiments the polymer further includes a block derived entirely or partially from an acrylamide. The acrylamide may be a poly(N-isopropylacrylamide), a polyacrylamide, a poly(hydroxymethylacrylamide, or any combination thereof.

In some embodiments the polymer may include a block derived entirely or partially from a methacrylamide. In some embodiments, the polymer may further include a block derived entirely or partially from an acrylate. In some embodiments, the polymer may further have a block derived entirely or partially from a methacrylate. In some embodiments, the polymer may further have a block derived entirely or partially from a vinyl monomer.

The polymer may be aggregated to form a micelle or a vesicle. In some embodiments, the polymer may further include polyethylene glycol.

The disclosure also includes compositions including: a block copolymer having monomer units of a boron moiety containing at least one pendant boronic acid moiety per repeat unit; and a therapeutic agent. The therapeutic agent may include a nucleic acid, a polynucletide, a peptide, a polypeptide, a protein, a pharmaceutical agent or any combinations thereof. The therapeutic agent may include a pharmaceutical formulation of insulin.

Compositions for monitoring the blood glucose levels of a mammal having at least one monomeric boron moiety wherein the boron moiety is incorporated pendantly or terminally are disclosed. Compositions for regulating blood glucose levels are of a mammal also disclosed.

Also included are methods for regulating blood glucose levels of a mammal including administering to a mammal in need thereof a pharmaceutical composition including: at least one monomeric boron moiety wherein the boron moiety is incorporated pendantly or terminally; and a pharmaceutical formulation of insulin.

Methods for preparing a polymer having at least one monomeric boron moiety wherein the boron moiety may be incorporated pendantly or terminally including: direct polymerization or copolymerization of boronic acid-containing monomers; and deprotection to boronic acid moieties are described in some embodiments.

Methods for preparing a polymer having at least one monomeric boron moiety wherein the boron moiety may be incorporated pendantly or terminally including: direct polymerization or copolymerization of boronic ester-containing monomers; and deprotection to boronic ester moieties are also described in some embodiments.

Methods for preparing a polymeric micelle or vesicle having at least one monomeric boron moiety wherein the boron moiety may be incorporated pendantly or terminally; and a pharmaceutical formulation including insulin; by a controlled/living polymerization method including: direct polymerization or copolymerization of boronic acid-containing monomers; deprotection to boronic acid moieties; and incorporating a pharmaceutical formulation including insulin into the polymer to form the polymeric nanoaggregate are described in some embodiments.

In some embodiments, methods for preparing a polymeric micelle or vesicle including at least one monomeric boron moiety wherein the boron moiety may be incorporated pendantly or terminally; and a pharmaceutical formulation comprising insulin; by controlled/living polymerization method including: direct polymerization or copolymerization of boronic ester-containing monomers; deprotection to boronic ester moieties; and incorporating a pharmaceutical formulation comprising insulin into the polymer to form the polymeric nanoaggregate are described.

The disclosure also describes methods for administration of the therapeutic agent compositions including: administering to a mammal a block copolymer micelle or vesicle including: at least one monomeric boron moiety wherein the boron moiety is incorporated pendantly or terminally; and a pharmaceutical formulation of the therapeutic agent; wherein the release of the therapeutic agent in the mammal includes dissolution of the block copolymer micelle or vesicle.

In some embodiments, the dissolution of the block polymer may be triggered by an increase in the local or global concentration in the mammal of a 1,2-diol or a 1,3-diol. The 1,2-diol or a 1,3-diol is a saccharide may be glucose, fructose, and sucrose.

The therapeutic agent release may be induced by dissolution of the block polymer micelles or vesicles triggered by an increase in the local or global concentration of a 1,2-diol or a 1,3-diol. In some embodiments, the therapeutic agent may be insulin.

Administration may include oral administration, sublingual administration, parenteral administration, topical administration, administration to eye or mucosal membranes. Parenteral administration may include intravenous, intraperitoneal, subcutaneous, intrathecal, injection to the spinal cord, intramuscular, intraarticular, portal vein injection, or intratumoral administration.

Also described are methods to remove boronic or boronate pinacol esters from boron-containing polymers or copolymers by treatment with a boronic acid-functionalized resin. Methods to polymerize boronic acid-containing monomers by reversible addition-fragmentation chain transfer polymerization and/or by reversible addition-fragmentation chain transfer polymerization are also set forth.

### BRIEF DESCRIPTION OF THE DRAWINGS

A better understanding of the present disclosure will be realized from the detailed description which follows, taken in conjunction with the accompanying drawings, in which example embodiments of the disclosure are illustrated.

FIG. 1 illustrates a reaction scheme, Scheme 1, in which Boronic acids (1) or boronates (3) may react with diols to form boronic esters (2) or boronate esters (4) in non-aqueous media or basic aqueous media, respectively.

FIG. 2 illustrates a reaction scheme, Scheme 2, showing synthesis of 3-Acrylamidophenylboronic Acid homo- and block copolymers by Reversible Addition-Fragmentation Chain Transfer (RAFT) Polymerization.

FIG. 3 shows various plots relating to RAFT homopolymerizations of 3-acrylamidophenylboronic acid (APBA, 1) at 70° C. in 95% DMF/5% water. FIG. 3A shows a pseudo first-order kinetic plot with selected molar ratios of monomer (M):chain transfer agent (CTA):initiator (I). FIG. 3B shows Mₙ, versus monomer conversion ([Mon]:[CTA]:[Init]=100:1:0.1).

FIG. 4 illustrates a reaction scheme, Scheme 3, relating to ionization and diol complexation equilibria of Boronic Acids in aqueous media.

FIG. 5A illustrates block copolymer self-assembly/dissociation in response to changes in pH or glucose concentration ([glucose]). FIG. 5B illustrates aqueous hydrodynamic size distributions of poly(3-acrylamidophenylboronic acid)-b-poly(N,N-dimethylacrylamide) (PAPBA₁₃₁-b-PDMA₁₃₈) as a function of pH and [glucose] at 25° C.

FIG. 6 shows ¹H NMR spectra of PAPBA macro-CTA in methanol-d₄ with residual DMF peaks removed.

FIG. 7 shows ¹H NMR spectra of PAPBA-b-PDMA in methanol-d₄ with residual ether peaks removed.

FIG. 8 shows ¹H NMR spectra for PAPBA-b-PDMA (A) before protection and (B) after protection (methanol-d₄). Residual DMF peaks are removed.

FIG. 9 shows normalized refractive index traces from size exclusion chromatography of PAPBA homopolymers.

FIG. 10 shows normalized refractive index traces from size exclusion chromatography of a PAPBA-b-PDMA block copolymer.

FIG. 11 illustrates a reaction scheme, Scheme 4, for synthesis of boronic ester and boronic acid (co)polymers by RAFT polymerization.

FIG. 12A shows a pseudo first-order kinetic plot and FIG. 12B shows a Mₙ, versus conversion both for RAFT of pBSt (5) with various ratios of monomer (Mon):chain transfer agent (CTA):initiator (Init). FIG. 12C shows SEC traces for a PpBSt homopolymer and block copolymer with PDMA. FIG. 12D shows a hydrodynamic diameter distribution for PpBSt₁₄₅-b-PDMA₂₇₃.

FIG. 13, Chart 1, depicts novel boronic acid-containing chain transfer agents CTAs 9, 10, 11, 12.

FIG. 14 depicts the hydrodynamic size of PAmPBA₁₃₁-b-PDMA₁₃₈ relating to self-assembly/dissociation in response to changes in pH or [glucose].

FIG. 15A illustrates a reaction scheme for boronic acid-terminal polymer reversibly complexing with a model diol in THF. FIG. 15B also provides a photograph of the reaction in which (1) is Alizarin red alone, (2) is Alizarin Red+PDMA-B(OH)₂, which is yellow and fluorescent, and (3) is Alizarin Red+PDMA-B(OH)₂+water (the product of hydrolysis of boronic ester), which is red.

FIG. 16 illustrates a reaction scheme, Scheme 5, which depicts aqueous ionization of boronic acid.

FIG. 17 illustrates a method for solution self-assembly and controlled release with diol-responsive block copolymers.

FIG. 18, Chart 2, depicts free or protected boronic acid monomers 9, 16, 17, 18.

### DETAILED DESCRIPTION

The present disclosure provides novel drug-delivery compositions including boronic acid-containing copolymers. In some embodiments, a composition may include a therapeutic agent including a nucleic acid, a polynucletide, a peptide, a polypeptide, a protein, a pharmaceutical or any combinations thereof. Methods for making a polymer nanoaggregate composition of boronic acid including at least one therapeutic agent (for example, insulin) as well as methods for administration of these agents are also set forth. Some embodiments of the disclosure may include compositions and methods for controlled drug delivery. Some embodiments of the disclosure may include compositions and methods for monitoring the blood glucose levels of a mammal. Some embodiments of the disclosure may include compositions and methods for regulating the blood glucose levels of a mammal.

Some embodiments of the disclosure may include nanoaggregates and/or nanoparticles of boronic-acid containing block copolymers, which may further include at least one therapeutic agent, and methods for preparing these copolymers by controlled/living polymerization methods. Exposure to an activating agent may induce controlled release of a therapeutic agent from nanoaggregates and/or nanoparticles. One example for the application of this technology is the controlled release of insulin within the bloodstream in response to a high concentration of blood glucose. Therefore, in some embodiments, the disclosure may include a treatment regimen for diabetes mellitus. In contrast to available treatment methods, some embodiments of the disclosure include simultaneously monitoring blood glucose and administering insulin by one feedback-controlled composition.

In some embodiments, a composition for treating Type II diabetes may include boron-polymer encapsulated insulin and/or other glucose-reducing drugs. Compositions and methods, according to some embodiments of the disclosure, may include site-specific administration and controlled-release of other therapeutics e.g., anticancer drugs, gene therapy agents, other peptide, polypeptide or protein therapeutic agents, antiviral agents, antibacterial agents, antifungal agents, and other glucose-reducing drugs (such as Pioglitazone, Glimepiride, Rosiglitazone, a bi-guanide, Metformin, chlorpropamide, glipizide, glyburide).

### Stimuli-Responsive Adaptive Boron-Containing Polymers

In some embodiments, the present disclosure may include novel stimuli-responsive and adaptive boron-containing polymeric compositions and methods for their synthesis by macromolecular engineering methodologies. Other boron-containing polymers have been shown to play a role in catalysis, separations, instilling flame retardancy, catalytic potential, pH-responsiveness, increased polarity, and sensing applications. Some biological applications of boronic-acid block copolymers have been reported (e.g., as lipase inhibitors to treat obesity (U.S. patent application Ser. No. 10/535,639); as agents to prevent tissue adhesions (U.S. Pat. No. 6,596,267); as coatings for contact lenses (US Published Application No. 2007 0116740); and in the areas of saccharide and nucleotide sensing). However, a limitation in the field of organoboron polymers is the lack of versatile synthetic techniques for the facile preparation of boronic acid (co)polymers with controlled architecture.

In one embodiment, the present disclosure may include controlled/living radical polymerization techniques for the synthesis of well-defined, highly functional polymers that respond via boronic acid/diol interaction chemistry. In these techniques, the robust chemistry of boronic acids may be utilized along with their ability for dynamic/reversible covalent bonding with 1,2- and 1,3-diols.

Boronic Acids and Reversible Diol Complexation

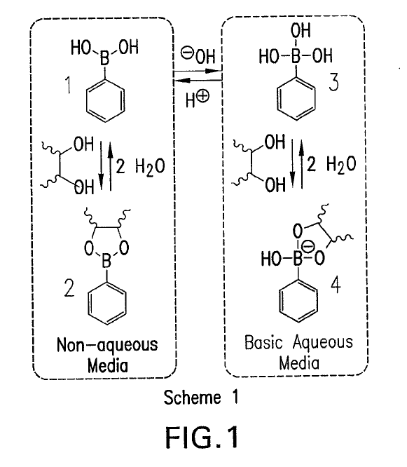

Boronic acids are compounds of the structure R—B(OH)₂, in which the trivalent boron atom contains an empty p orbital. The resulting two electron deficiency leads to mild Lewis acidity and interesting complexation behavior. Boronic acids have been exploited as useful intermediates in a variety of organic reactions, most notably Pd-catalyzed Suzuki-Miyaura coupling. Prominence of boronic acids in small molecule synthesis has led to a wide variety of commercially available examples. They are also characterized by benign degradation products and low toxicity. The ability of boronic acids to form cyclic boronic or boronate esters upon reaction with 1,2-diols and/or 1,3-diols is a feature of their chemistry exploited in embodiments of the current disclosure. (See FIG. 1, Scheme 1). In aqueous media above the pKₐ of the boronic acid (1), the tetracoordinate boronate species (3) demonstrates high affinity for diols, resulting in cyclic boronate esters (4). These properties have been utilized for saccharide sensing because of efficient complexation with cyclic sugars that contain cis 1,2-diols, including glucose, fructose, mannose, etc. The affinity of the interaction has been employed to prepare carbohydrate and nucleotide transporters and to mimic antibodies targeted for cell surface carbohydrates. Boronic acids also reversibly form trigonal planar boronic esters (2) under anhydrous conditions. Thus, the reversible and covalent nature of boronic ester formation is versatile. Several factors affect the affinity of binding, including sterics, boronic acid pKₐ, and diol acidity.

Two particular aspects of the boronic acid-diol complexation phenomenon are used in some embodiments of the present disclosure. First, complexation in aqueous media results in a significant pKₐ reduction of the boronic acid. Therefore, as the concentration of diol increases, the equilibrium between neutral boronic acid and anionic hydroxyboronate shifts to favor the anionic species. This results in increased hydrophilicity of the boronic acid compound, thereby imparting stimuli (diol) responsive solubility. Second, boronic acid-diol complexes are reversible covalent species with characteristics of supramolecular systems. As compared to supramolecular assemblies that rely on intermolecular forces, boronic ester formation occurs via stable covalent bonding, while still maintaining the potential for dynamics, self-assembly, and self-repair. In some embodiments, the present disclosure utilizes the high affinity, reversibility, and selectivity of the boronic-acid-diol interactions to generate stimuli-responsive block copolymer assemblies by controlled polymer synthesis and macromolecular assembly methods.

Boronic Acids Macromolecular Chemistry

In some embodiments, boron functionality may be introduced to a composition by post-polymerization modification of precursor polymers or by the direct polymerization of boron-containing monomers. A variety of synthetic techniques may be employed to prepare boron-containing polymers, including condensation, coordination, ring-opening metathesis, and conventional radical polymerizations. Conventional radical polymerization methods provide polymers with pendant boron functionality due to facile experimental setup and lack of significant side reactions. However, conventional radical polymerization methods typically result in polymers with unpredictable molecular weights, broad molecular weight distributions, and no significant end group control generally precluding block copolymer formation. End group control may be especially important, as it directly limits the ability to prepare complex copolymer architectures that self-assemble in solution. Thus, conventional radical polymerization methods are limited to producing uncontrolled random copolymers of acrylamido and boron-containing monomers.

Controlled/living radical polymerization (CRP) facilitates the preparation of (co)polymers with predetermined molecular weights, narrow molecular weight distributions, and high degrees of chain end functionality. While resulting in control comparable to living ionic polymerizations, CRP may be conducted under less stringent conditions and may demonstrate enhanced functional group tolerance. Accordingly, organoboron vinyl (co)polymers have been prepared (by Jäkle and coworkers), via atom transfer radical polymerization (ATRP), either from silylated precursors that were subsequently borylated with BBr₃ or from the polymerization of organoboron monomers. The control afforded by ATRP gives (co)polymers with predetermined molecular weights and narrow molecular weight distributions. Bulk phase separation of polystyrene-b-poly(4-pinacolatoborylstyrene) and conveniently manipulated Lewis acidity of substituted polymeric boronic esters have been demonstrated.

### Biological Relevance of the Compositions

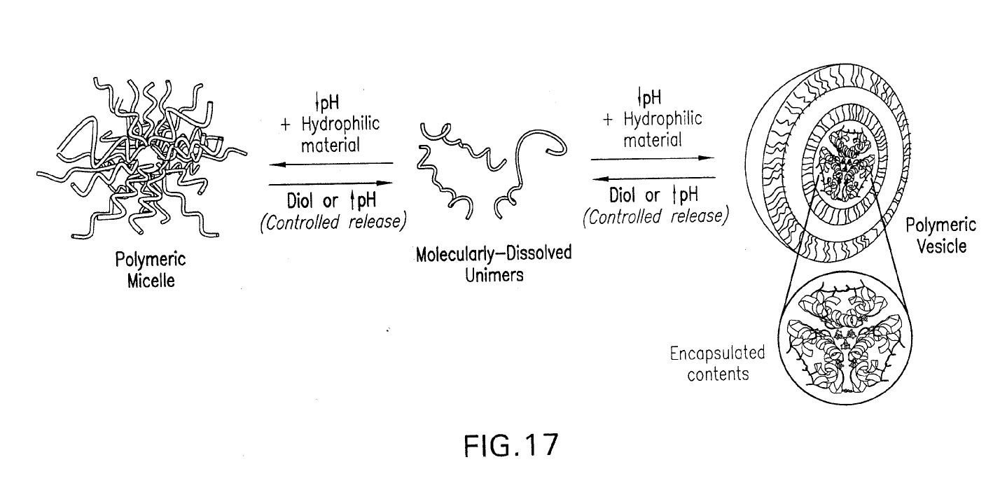

The present disclosure may include, according to some embodiments, methods to design and synthesize novel boronic acid-containing block copolymers that reversibly self-assemble or dissociate in response to biologically-relevant molecules to form or disassemble micelles and vesicles with tunable size, morphology, and controlled release potential. (See FIG. 17). While traditional stimuli employed to induce block copolymer assembly in water include changes in pH, salt concentration, or temperature, representing a rather narrow range of potential triggering mechanisms, solution aggregation may be induced, according to some embodiments, by specific naturally occurring molecules. For example, naturally occurring diols like glucose, fructose, and adenosine, may trigger solubility transformations in responsive boronic acid-containing blocks.

In some embodiments, boronic acid polymeric compositions may include at least one therapeutic agent. A therapeutic agent may be any agent described in the specification or known in the art including peptides, polypeptides, proteins, hormones, steroids, nucleic acids, chemical drugs, pharmaceuticals. A composition, in some embodiments, may include additional agents such as buffers, co-enzymes, metallic components, ions or any other molecule desired and/or required for the therapeutic agent (e.g., to have optimum biological function and/or to be stable).

In some embodiments, a composition may be assembled and/or aggregated into vesicles or micelles. In some embodiments, vesicles or micelles of the disclosure may be biocompatibile. Therefore, the outer material of the vesicle/micelles may include biocompatible polymers, may be non-immunogenic, may be stable in the bloodstream, may be non-toxic, may be capable of targeted delivery at a specific tissue, organ or cell in the body, and/or may have desired pharmacokinetics.

In addition to utility as drug delivery agents, the nanostructures of the disclosure may be used as affinity ligands for separation of carbohydrates and glycoproteins, as antibody mimics targeted for cell-surface carbohydrates, and for the delivery of nucleotides.

### Application for Diabetes

Given the prevalence and rapid growth of diabetes throughout the world, a significant need exists for alternative treatment options. Diabetes is characterized by high concentrations of glucose in the blood. Currently, the major treatment option permitted by the FDA is frequent subcutaneous injection of insulin. For example, a significant limitation in managing type I diabetes mellitus is lack of feed-back controlled insulin release mechanisms. A need exists for effective systems that automatically release insulin in response to high glucose concentrations. A composition able to perform this function may include, for example, biocompatible micelles and/or vesicles having boronic acid-containing block copolymers that may include, for example, insulin as a therapeutic agent. This composition may also be able to reversibly self-assemble in response to diols such as glucose, thereby sensing high glucose concentrations in the blood stream and responsively disassembling to deliver the therapeutic agent, e.g., insulin. (See FIG. 17).

The specific concentration that constitutes a high glucose concentration for certain embodiments of this disclosure may include glucose concentrations at which additional glucose is needed by the body in order to remove sugar from the blood stream, concentrations considered clinically to be high, or concentrations at which administration of insulin is clinically indicated. For example, high glucose concentrations may include concentrations indicated to be high by current at home or clinical glucose testing technology. In one example, high glucose concentrations may include concentrations at which an alarm is triggered by at home glucose test strips or subdermal sensors. In one example, a high blood glucose concentration may be more than 180 mg/dL, more than 200 mg/dL, or more than 240 mg/dL, or more than 500 mg/dL. Depending on the mode or location of administration of compositions of the current disclosure, the relevant glucose concentration may be in a different bodily fluid, such as interstitial fluid, and may differ from glucose concentrations considered high in the blood as known to one skilled in the art. In certain embodiments, a glucose concentration considered high for release of the drug to be desirable may differ depending on the effects of the drug and the time needed to cause such effects as well as other clinical considerations.

Thus, according to some embodiments, following oral ingestion and/or injection, nanoscale polymer assemblies may detect high concentrations of glucose in the bloodstream and automatically release insulin. These nanosized drug-delivery vehicles may be orally ingested or injected into the bloodstream at less frequent intervals (as compared to insulin) and combine the glucose monitoring and insulin delivery process. According to some embodiments, nanoscale insulin molecules may be approximately about 6 nm in size and the polymeric aggregates may be about 10-500 nm in diameter. In some embodiments, polymeric aggregates may be 100-200 nm in diameter. In some embodiments, the polymeric aggregates may be 20-200 nm. In some embodiments, when dissolved in an aqueous system (e.g., the human bloodstream), the polymers self-assemble into tiny, hollow spheres called vesicles. One segment of the block copolymers may bind to glucose molecules, which in turn triggers disruption or dissolution of the vesicle membrane such that the encapsulated insulin is released, in a manner similar to a balloon popping.

In this approach, both glucose monitoring and insulin release may be combined into one feedback-controlled system that may requires reduced patient vigilance, thereby potentially increasing compliance and diabetes management. The vesicles may be designed to be injected or orally ingested and subsequently reside in the bloodstream until insulin release is automatically dictated by an increase in the surrounding concentration of glucose to a level considered high for that particular glucose composition.

Because the vesicles are (a) passivated with a biocompatible, non-immunogenic shell and (b) larger than free insulin, the vesicles may experience increased blood residency times compared to insulin or other therapeutics and thus the frequency of administration may also be reduced. According to one embodiment, the composition may include a vehicle for the delivery of exogenous therapeutic agents to cells and/or tissues which are safe to use, easy to produce in large quantity and have sufficient stability and safety to be practicable as a pharmaceutical.

The ability afforded by RAFT polymerization to accurately control polymer molecular weight may be particularly useful for these applications in vivo. The vesicles or micelles of the disclosure may have total aggregate molecular weights larger than approximately 45 kDa to avoid glomerular excretion by the kidney. Moreover, the actual hydrodynamic size of the vesicles may depend on, in addition to other factors, the molecular weight of their constituent block copolymers. In some embodiments, target vesicles may be 10-1000 nm. In some embodiments, vesicles greater than 10 nm but smaller than 200 nm to help avoid detection by the reticuloendothelial system recognition are contemplated. In some embodiments, the block copolymers composing the polymeric vesicles may be of molecular weight 1-200 kDa.

For prolonged blood residency of polymeric carriers, factors such as size and surface character determine clearance kinetics and distribution in a biological milieu. Particles smaller than approximately 5 μm or which contain relatively hydrophobic surfaces may be rapidly removed to the liver and spleen macrophages. In some embodiments, steric stabilization with hydrophilic, non-ionic polymers may be used to increase the biostability of circulating nanoparticles. Without wishing to be bound to any theory, the enhanced stability provided by nanoparticle steric stabilization may arise from the polymer coating conferring surface invisibility that prevents the adsorption of various blood components (e.g., opsonin ligands) and adherence to the blood vessel endothelium. Steric stabilization may confer a relative ‘invisibility’ to the colloidal particles, which may be reflected by a reduced uptake by liver and spleen macrophages and extended blood circulation times. PEG polymers may be used to provide steric stabilization to nanoparticles in vivo. Liposomes composed of small molecule surfactants may also be used to increase blood circulation half-lives to 48 hours or more. In some embodiments PEG-coated polymeric compositions may be used. In some embodiments the PEG polymers may have molecular weights of at least 1900 Da to about 5000 Da. Thus PEG or liposomes may be used to construct macroCTA polymers of the disclosure. PEG may also line the interior wall by virtue of the vesicle's bilayer morphology. This feature may increase the resistance to protein adsorption, and thereby may prevent denaturation of the encapsulated insulin.

Based on the variation in circulatory life time the engineered long-circulating nanoparticles, in some embodiments the nanoparticles may circulate for 3-5 days. In other embodiments they may circulate from 2-10 days, or from 3-15 days. In some embodiments, the total number of insulin injections may be reduced from several times per day to once every 1-3 days, or once every 2-10 days, or once every 3 days, or once every 4 days or once every 5 days. Provided that such polymeric vesicles have sufficient amounts of glucose-responsive insulin, the number of required insulin injections for diabetics may be reduced.

Additionally, some compositions of the current disclosure may be highly specific to glucose and carbohydrates and thus not as sensitive to inaccuracies in glucose detection as other available detection methods. For example, hand held electrochemical sensors currently popular for diabetes management may report incorrect glucose levels in patients who have high levels of Vitamin C (Ascorbic Acid) or acetominophen (commonly sold as Tylenol®) in their blood because these chemicals may electrochemically mimic glucose detection molecules. In contrast, some compositions of the current disclosure may not inappropriately respond to other chemicals commonly found in the blood. This more precise response ability may be due, in some embodiments, to a direct and more specific glucose recognition system than is used in other glucose detection methods.

### Pharmaceuticals

Pharmaceutical compositions may include combinations of an active therapeutic agent with a carrier, inert or active, making the composition suitable for diagnostic or therapeutic use in vitro, in vivo or ex vivo. A pharmaceutically acceptable carrier may encompasses any of the standard pharmaceutical carriers, such as a phosphate buffered saline solution, water, and emulsions, such as an oil/water or water/oil emulsion, and various types of wetting agents. The compositions also may include stabilizers and preservatives. For examples of carriers, stabilizers and adjuvants, see Martin, REMINGTON's PHARM. SCI., 15th Ed. (Mack Publ. Co., Easton (1975), incorporated in relevant part herein. An effective amount may be an amount sufficient to effect beneficial or desired results. An effective amount may be administered in one or more administrations, applications or dosages. Mammals may include, but are not limited to, humans, murines, simians, farm animals, sport animals, and pets.

### Methods of Administration

Administration or delivery of any therapeutic composition of the present disclosure may include any method which ultimately provides the therapeutic agent to the cell/tissue or site where it is needed. Examples include, but are not limited to, oral ingestion, sublingual administration, subcutaneous injection, intravenous administration, parenteral administration or topical application. Topical administration may include administration to eye or mucosal membranes. In some embodiments, pharmaceutical compositions may be administered parenterally, i.e., intravenously, intraperitoneally, subcutaneously, intrathecally, injection to the spinal cord, intramuscularly, intraarticularly, portal vein injection, or intratumorally. In other embodiments, pharmaceutical preparations may be contacted with a target tissue by direct application of the preparation to the tissue.

Administration in vivo may be effected in one dose, continuously or intermittently throughout the course of treatment. During the initial determination of dosage requirements, monitoring may be advisable to ensure that the composition is having its desired effect or not creating adverse side effects. For example, in the case of compositions for treatment of diabetes, blood sugar monitoring may be advisable to ensure that the dosage is causing an adequate, but not too drastic, decrease in blood sugar. Methods of determining the most effective means and dosage of administration are known to those of skill in the art, in light of this disclosure, and may vary with the composition used for therapy, the purpose of the therapy, the target cell or tissue being treated, and the mammal being treated. Single or multiple administrations may be carried out with the dose level and pattern being selected by the treating physician. Suitable dosage formulations and methods of administering the agents may be empirically determined by those of skill in the art in light of this disclosure.

Administration in vivo may occur by more than one distinct step. For example, the boronic acid polymer nanoaggregates carrying the agent to be delivered may be introduced into the body, but delivery or release of the agent may not occur until the nanoaggregates is induced to dissociate by a separately administered activating agent (e.g., a 1,2- or 1,3-diol). The activating agent may be administered parenterally, i.e., intravenously, intraperitoneally, subcutaneously, intrathecally, injection to the spinal cord, intramuscularly, intraarticularly, portal vein injection, intratumorally, or by absorption through the skin, mucous membranes, or eyes.

The agents and compositions of the present disclosure may be used in the manufacture of medicaments and for the treatment of humans and other animals by administration in accordance with conventional procedures, such as an active ingredient in pharmaceutical compositions.

While it is possible for the compositions of the disclosure to be administered alone, it may be preferable to present them as a pharmaceutical formulation including at least one active ingredient (e.g., a boron-containing micelle and/or vesicle comprising insulin) together with one or more pharmaceutically acceptable carriers therefore and optionally other therapeutic agents. According to some embodiments, a carrier may be acceptable in the sense of being compatible with the other ingredients of the formulation and not injurious to the mammal.

### EXAMPLES

The present disclosure may be better understood through reference to the following examples. These examples are included to describe exemplary embodiments only and should not be interpreted to encompass the entire breadth of the invention.

### Example 1

### Controlled Polymerization of Organoboron Monomers

A current limitation in the field of organoboron polymers is the lack of versatile synthetic techniques for the facile preparation of boronic acid (co)polymers with controlled architecture. The present disclosure, in some embodiments, provides new methods of controlled polymer synthesis. Synthesis and aqueous solution behavior of amphiphilic organoboron block copolymers, especially those with acrylamido hydrophilic blocks has been used. While the success of ATRP for the polymerization of most acrylamido monomers has dramatically improved, reversible addition-fragmentation chain transfer (RAFT) polymerization techniques have been used for the synthesis of a range of polyacrylamides. RAFT may be conducted under relatively mild conditions, may be applicable to nearly any monomer susceptible to radical polymerization, and may employed to prepare a range of well-defined complex macromolecular topologies. In some embodiments, stimuli-responsive and water-soluble acrylamido boron-containing polymers have been synthesized.

Employing RAFT polymerization, boronic acid-containing homopolymers and block copolymers with poly(N,N-dimethylacryl-amide)(PDMA) were prepared by the present methods. In some embodiments, the methodology may include (i) direct polymerization of free, unprotected boronic acid monomers; or (ii) polymerization of boronic ester monomers followed by subsequent deprotection to yield boronic acid-containing polymers.

Materials Used

2-Dodecylsulfanylthiocarbonylsulfanyl-2-methylpropionic acid (DMP) chain transfer agent (CTA) was prepared. N,N-Dimethylacrylamide (DMA, Fluka, 98%) was passed through a small column of basic alumina for catalyst removal prior to polymerization. 2,2′-Azobisisobutyronitrile (AIBN, Sigma, 98%) was recrystallized from ethanol. 3-Aminophenyl boronic acid (Boron Molecular), acryloyl chloride (Alfa Aesar, 96%), 1,3,5-trioxane (Acros Organics, 99.5%), pinacol (Acros Organics, 99%), sodium hydrogen carbonate (Acros Organics, 99.5%), sodium hydroxide pearl (Alfa Aesar, 97%), hydrochloric acid (Alfa Aesar, 36% (w/w) aq. solution), D-glucose (Mallinckrodt), N,N-dimethylformamide (DMF) (Aldrich 99.9%), tetrahydrofuran (THF) (Acros Organics, 99.9%), diethyl ether, dimethylsulfoxide-d₆ (DMSO-d₆, Cambridge Isotope, 99.9% D), CDCl₃ (Cambridge Isotope, 99% D), and methanol-d₄ (Cambridge Isotope, 99.8% D) were used.

Analyses

GPC was conducted in DMF (with 0.05 M LiBr) at 55° C. with a flow rate of 1.0 mL/min (Viscotek GPC Pump; Columns: ViscoGel I-Series G3000 and G4000 mixed bed columns: molecular weight range 0-60×10³ and 0-400×10³ g/mol, respectively). Detection consisted of a Viscotek refractive index detector operating at γ=660 nm, a Viscotek UV-Vis detector operating at γ=254 nm, and a Viscotek Model 270 Series Platform, consisting of a laser light scattering detector (operating at 3 mW, γ=670 nm with detection angles of 7° and 90°) and a four capillary viscometer. Molecular weights were determined by the triple detection method. ¹H NMR spectroscopy was conducted with a Bruker Avance 400 spectrometer operating at 400 MHz. Dynamic light scattering was conducted with a Malvern Zetasizer Nano-S equipped with a 4 mW, 633 nm He—Ne laser and an Avalanche photodiode detector at an angle of 173°.

(i) Direct Polymerization of Free, Unprotected Boronic Acid Monomers

Well-defined boronic acid (co)polymers were prepared by direct controlled polymerization of free, unprotected boronic acid monomers using a mechanism suitable for controlling the polymerization of functional Lewis acidic monomers. RAFT homo- and block (co)polymerization of a free boronic acid acrylamido monomer and solution properties of amphiphilic block copolymers that result from copolymerization of a free boronic acid monomer with a hydrophilic monomer are described herein. Several examples of stimuli-responsive block copolymers includes e.g., temperature-responsive systems, the block copolymers described herein self-assemble/dissociate in response to changes in pH and, the concentration of diols in the surrounding medium. Thus, saccharide-responsive block copolymers are described.

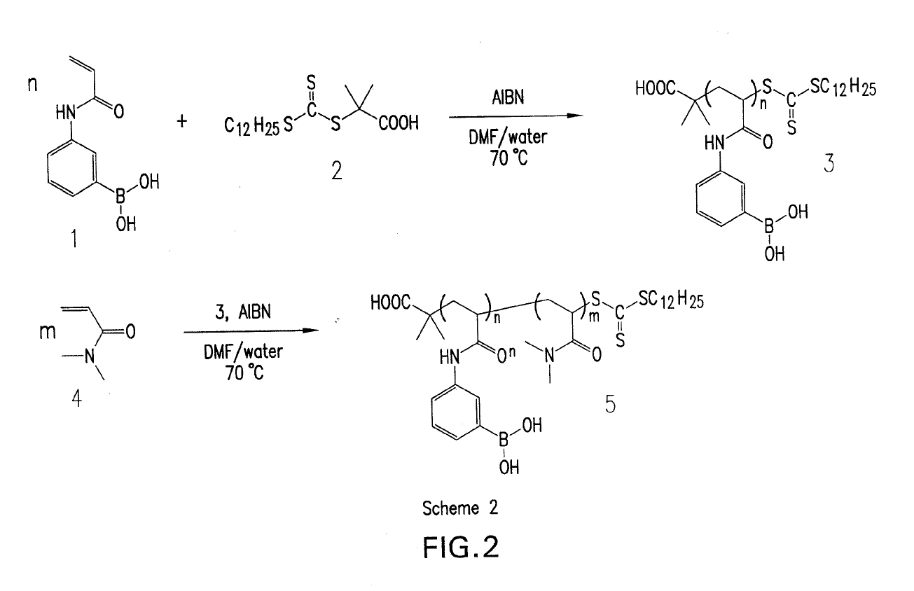

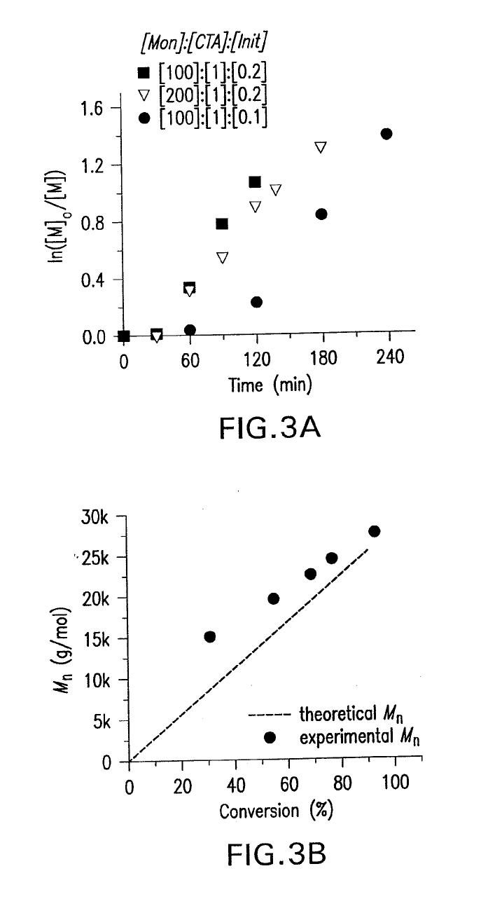

The RAFT polymerization was based on functional group tolerance and particular applicability for the synthesis of well-defined, water-soluble acrylamido polymers. As depicted in FIG. 2, Scheme 2,3-Acrylamidophenylboronic acid (APBA, 1) was polymerized with 2-dodecylsulfanylthiocarbonylsulfanyl-2-methylpropionic acid (DMP, 2) as the chain transfer agent (CTA) and 2,2′-azobisisobutyronitrile (AIBN) as the initiator at 70° C. in 95% DMF/5% water. The molar ratio of [monomer]:[CTA]:[initiator] was varied to observe the effect on polymerization kinetics and molecular weight control. After a brief inhibition period, pseudo first-order kinetics were observed up to high conversion (FIG. 3A). Molecular weight analysis by size exclusion chromatography (SEC) necessitated protection of the boronic acid residues by esterification with pinacol, after which good agreement between theoretical and experimental molecular weights was observed. For instance, with [monomer]:[CTA]:[initiator]=[100]:[1]:[0.2], 67% conversion was obtained in 150 min, resulting in polymer with Mₙ=19,200 g/mol (M_{w}/Mₙ=1.13), in good agreement with the theoretical Mₙ of 18,500 g/mol. During polymerization, the Mₙ of poly(3-acrylamido-phenylboronic acid) (PAPBA, 3) increased linearly with a slight deviation at low conversion, potentially due to inefficient chain transfer early in the polymerization (FIG. 3B). Despite this, the molecular weight distributions for the polymers remained narrow (M_{w}/Mₙ=1.04-1.16) throughout the polymerizations (Table 1).

TABLE 1
Reversible Addition-Fragmentation Chain Transfer (RAFT) Homo-
and Block Copolymerization of 3-Acrylamidophenylboronic
Acid (APBA, 1) at 70° C.

| | | Conv.^{b} | M_{n,theo.}^{b} | M_{n.}^{c} | |
|---|---|---|---|---|---|
| | [M]:[CTA]:[I]^{a} | (%) | (g/mol) | (g/mol) | M_{w}/Mₙ^{c} |
| PAPBA | [100]:[1]:[0.1] | 71 | 19 500 | 19 700 | 1.16 |
| PAPBA | [100]:[1]:[0.2] | 67 | 18 500 | 19 200 | 1.13 |
| PAPBA | [200]:[1]:[0.1] | 74 | 40 600 | 37 800 | 1.16 |
| PAPBA-b-PDMA | [100]:[1]:[0.2] | 96 | 35 000 | 38 700 | 1.17 |

PAPBA-b-PDMA: PAPBA-b-poly(N,N-dimethylacrylamide)
^{a}Molar ratio of monomer (M):chain transfer agent (CTA):initiator (I).
^{b}Determined by ¹H NMR spectroscopy (theoretical molecular weights (M_{n,theo}) calculated assuming 100% protection with pinacol).
^{c}Determined by SEC of the pinacol-protected polymers.

PAPBA homopolymers were used as macro-chain transfer agents (macroCTAs) to synthesize diblock copolymers with N,N-dimethylacrylamide (DMA, 4). ¹H NMR and SEC analyses of the resulting block copolymer confirmed successful incorporation of DMA. After copolymerization of DMA with a PAPBA macroCTA (3) of Mₙ=25,000 g/mol, the molecular weight of the resulting block copolymer increased to Mₙ=38,700 g/mol (M_{n,theory}=35,000 g/mol), and a new signal in the ¹H NMR spectrum was observed at δ=2.93 ppm, arising from the methyl groups of the poly(N,N-dimethylacrylamide)(PDMA) units. Block copolymer formation was confirmed by comparison of the SEC molecular weight distributions described in sections below, though low molecular weight tailing may indicate either the presence of a small amount of dead macroCTA or inefficient pinacol protection prior to analysis. Nonetheless, the polydispersity index (M_{w}/Mₙ) of the resulting block copolymer remained below 1.2. Block copolymer compositions calculated with data from both ¹H NMR and SEC were in good agreement.

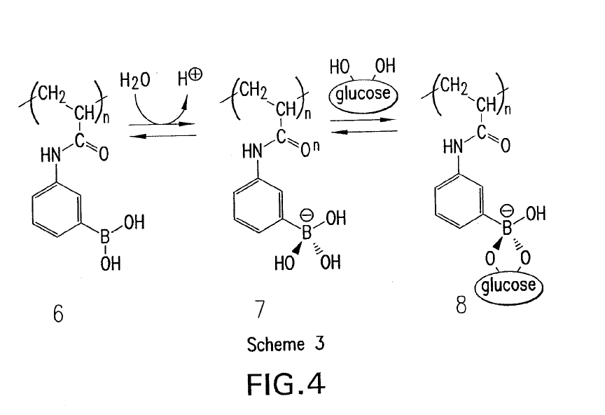

The solution behavior of the resulting block copolymers was also studied. Boronic acids are uniquely stimuli-responsive, in that their water solubility is tunable by changes in both pH and solution diol concentration, the latter of which has led to boronic acids being exploited as saccharide receptors. In aqueous media, boronic acids exist in equilibrium between forms that are neutral (typically insoluble) (6) and anionic (soluble) (7) (FIG. 4, Scheme 3). Cyclic ester complexes between 6 and 1,2- or 1,3-diols are usually hydrolytically unstable, but 7 readily forms boronate esters (8) in the presence of vicinal diols (FIG. 4, Scheme 3). An increase in concentration of 8 shifts the ionization equilibria, effectively lowering the pKₐ of the acid (FIG. 4, Scheme 3). Thus, complexation adjusts the overall equilibrium from neutral/insoluble boronic acid moieties to anionic/hydrophilic boronates. Therefore, the extent of ionization (and water solubility) of boronic acid-containing polymers increases with diol concentration. This ability was used to induce self-assembly of the PAPBA-b-PDMA block copolymers at pH<pKₐ of the boronic acid, and the subsequent diol (glucose)-dependent solubility was used to trigger aggregate dissociation.

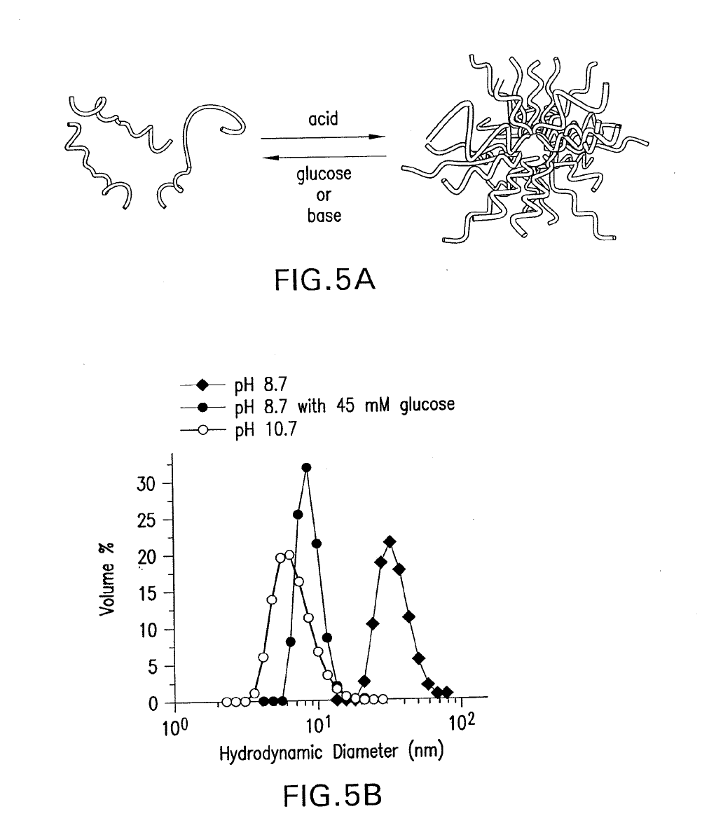

Dynamic light scattering (DLS) was employed to investigate the solution behavior of the double-hydrophilic block copolymers. PAPBA-b-PDMA may be both pH- and diol-sensitive. pH sensitivity arises as a result of the responsive organoboron block remaining soluble above the pKₐ of its boronic acid moieties. PAPBA₁₃₁-b-PDMA₁₃₈ was dissolved at pH 10.7 to give unimers with a hydrodynamic diameter (Dₕ) of approximately 7 nm (FIG. 5B). When the pH was slowly reduced below the pKₐ of the PAPBA block (pKₐ≈9) by dialysis against deionized water, self-assembly leads to formation of micelles. Indeed, aggregates with an average hydrodynamic diameter of 35 nm were observed by DLS (FIGS. 5A & 5B). Aggregates micelles may be composed of a hydrophilic PDMA corona and a hydrophobic PAPBA core.

In addition to pH susceptibility, response of PAPBA-b-PDMA to concentration of diols in the surrounding medium was analyzed. Upon the addition of glucose to yield a final solution concentration of [glucose]=45 mM (pH=8.7), the average hydrodynamic diameter dramatically decreased to 9 nm, indicative of aggregate disassembly. Under these conditions, cyclic boronate ester formation between glucose and the boronic acid moieties of the PAPBA block led to both blocks of PAPBA-b-PDMA being soluble (FIG. 5B).

The ability to prepare well-defined boronic acid-containing (co)polymers without resorting to protection/deprotection strategies may be used to prepare controlled topology organoboron polymers in a variety of biological and catalytic applications. A facile method to prepare block copolymers via direct RAFT polymerization of unprotected boronic acid monomers have been described. In addition to expanding the range of functionality that can be directly incorporated into well-defined polymers, this method provides simplified access to a new class of “smart” block copolymers that may demonstrate unique pH-, sugar-responsive self-assembly.

### Synthesis of 3-acrylamidophenylboronic Acid Monomer (APBA)

APBA was prepared by a method derived from Shinkai et al. 3-Aminophenylboronic acid (3.0 g, 0.022 mol) was dissolved in a 1:1 mixture of THF (40 mL) and water (40 mL) in a round bottom flask. Sodium hydrogen carbonate (3.7 g, 0.044 mol) and acryloyl chloride (4.0 g, 0.044 mol) were added to the flask at 0-5° C. The solution was stirred for 4 h and THF was subsequently evaporated. A solid crude product was obtained and stirred in ethyl acetate for 2 h. After filtering the solid materials, the ethyl acetate layer was washed with water (50 mL), saturated sodium bicarbonate solution (50 mL), water (50 mL) and brine (50 mL). The ethyl acetate layer was concentrated under reduced pressure providing the 3.5 g of orange solid in 84% yield (4.17 g=100% product). Further, the purification of monomer was carried out via the recrystallization from hot water three times. ¹H NMR (δ, ppm)(400 MHz, DMSO-d₆): 10.07 (s, 1H, NH), 8.00 (s, 2H, B(OH)₂), 7.89, 7.83-7.81, 7.51-7.49, 7.31-7.29 (s, d, d, t, 1H each, ArH), 6.46-6.42, 6.27-6.22 (2d, dd, 1H each, vinyl CH₂), 5.75-5.72 (dd, 1H, vinyl CH).

RAFT Homopolymerizations of APBA

RAFT polymerization of APBA was carried out as follows. APBA (1.50 g, 7.9 mmol), DMP (0.028 g, 0.079 mmol), AIBN (0.86 mg, 0.0079 mmol), and trioxane (35 mg, 0.39 mmol) (as an internal standard) were dissolved in 95/5 DMF/water (15 mL) in a sealed 20 mL vial. The molar ratio of [APBA]:[CTA]:[AIBN] was 100:1:0.1. The sealed vial was deoxygenated with nitrogen for approximately 30 min and then placed in a preheated reaction block at 70° C. Samples were removed periodically by syringe to determine molecular weight, polydispersity index (PDI), and monomer conversion by SEC and ¹H NMR spectroscopy. Methanol-d₄ was used as the solvent for ¹H NMR spectroscopy.

Raft Block Copolymerizations of N,N-dimethylacrylamide (DMA) with a PAPBA Macro-Chain Transfer Agent (Macro CTA)

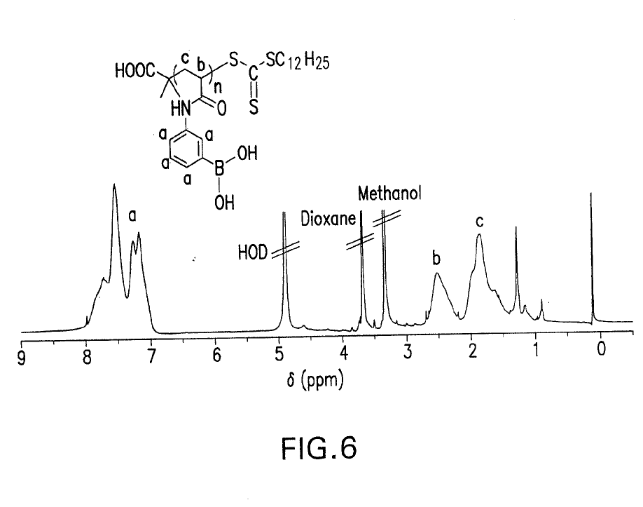

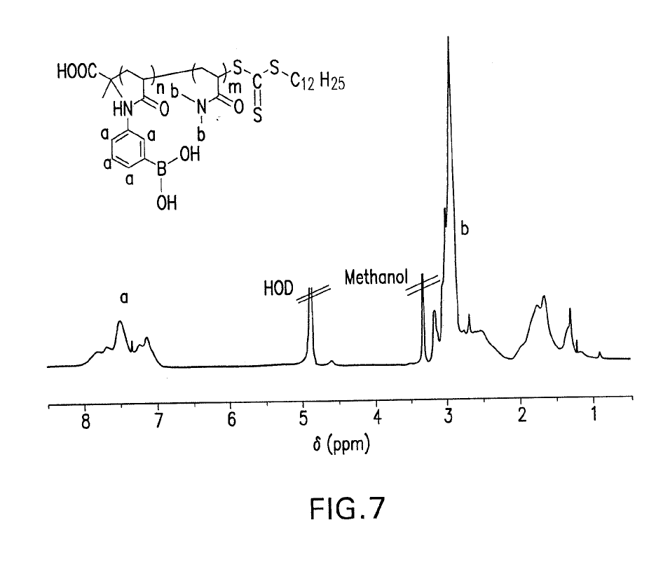

RAFT block copolymerization was carried out as follows. DMA (0.25 g, 2.5 mmol), PAPBA macro CTA (M_{n,unprotected}=17,900 g/mol, M_{n,protected}=25,000 g/mol, M_{w}/Mₙ=1.09) (0.45 g, 0.025 mmol), AIBN (0.83 mg, 0.0051 mmol), and trioxane (11.5 mg) were dissolved in 95/5 DMF/water (2 mL) in a sealed 20 mL vial. The molar ratio of [APBA]:[CTA]: [AIBN] was 100:1:0.2. The solution was deoxygenated with nitrogen for approximately 30 min and then placed in a preheated reaction block at 70° C. The polymerization was quenched after 20 h by removing the polymerization vial from the heating block and exposing the reaction solution to air. The resulting PAPBA-b-PDMA (96% conversion; block composition calculated by SEC: PAPBA=49%, and PDMA=51%; block composition calculated by ¹H NMR spectroscopy (integration of aromatic protons (C₆H₄) from 7-8 ppm of the PAPBA block compared to dimethyl protons (CH₃) at 2.93 ppm of the PDMA block): PAPBA=56%, and PDMA=44%; M_{n,protected}=38,700 g/mol; M_{w}/Mₙ=1.17) was isolated by precipitating into diethyl ether, filtering, and drying under vacuum. Methanol-d₄ was used as the solvent for ¹H NMR spectroscopy. A new peak at 2.93 ppm was observed for the block copolymer, confirming the presence of the PDMA units (CH₃ group)(FIG. 6 and FIG. 7).

### General Protection Procedure for PAPBA and PAPBA-b-PDMA

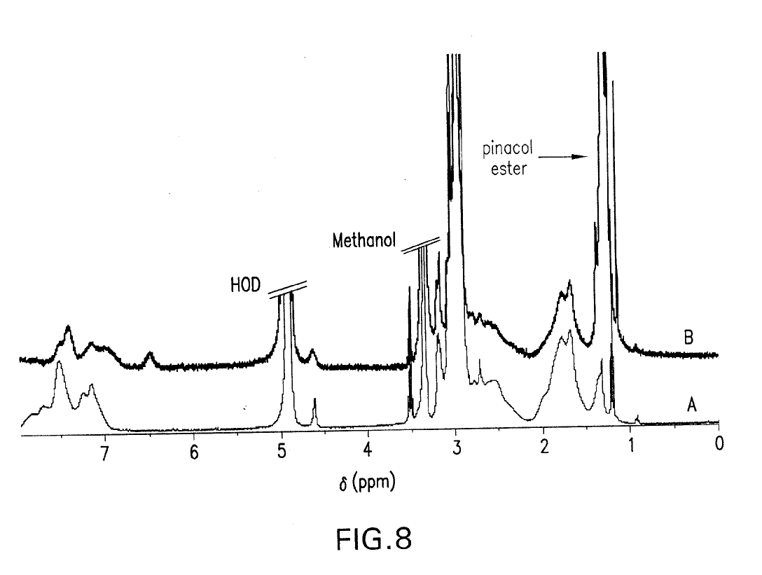

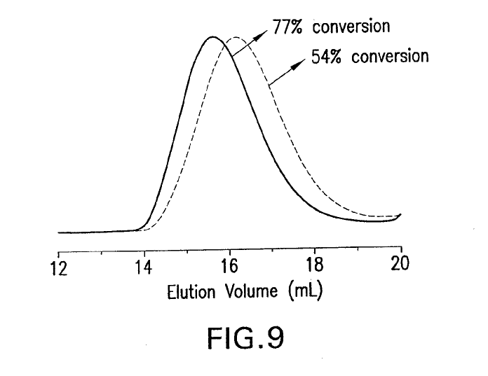

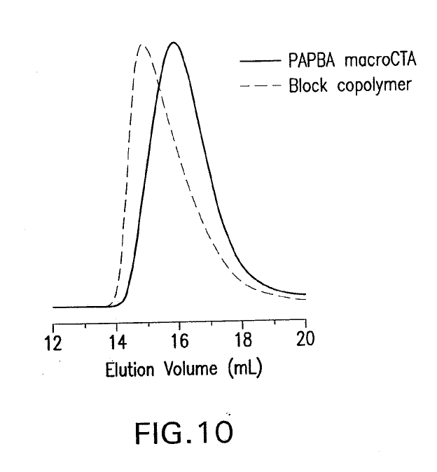

To facilitate analysis of the APBA (co)polymers by SEC, the boronic acid residues were protected with pinacol. A typical protection procedure is as follows. PAPBA-b-PDMA (0.10 g, 0.52 mmol), pinacol (0.56 g, 4.7 mmol), and molecular sieves were placed in a Schlenk flask. Anhydrous DMF (10 mL) was added, and the mixture was stirred under N₂ at 105° C. for 16 h. The mixture was filtered, and the protected (co)polymer was precipitated into cold diethyl ether. Successful protection was confirmed via ¹H NMR spectroscopy by the appearance of pinacol ester methyl protons of protected PAPBA-b-PDMA at δ=1.26 ppm (FIG. 8). SEC analyses show an increase in molecular weight with conversion for PAPBA homopolymers and good blocking efficiency for PAPBA-b-PDMA (FIG. 9 and FIG. 10).

### Dynamic Light Scattering (DLS) Measurements of PAPBA-b-PDMA

A 0.04% weight solution of PAPBA-b-PDMA (4.2 mg, Mₙ=38,700 g/mol, M_{w}/Mₙ=1.17) in basic water (10 mL, pH≈11.0) was placed in 3,500 MWCO dialysis tubing and dialyzed 48 h against deionized water with constant stirring. The resulting aqueous solution was sonicated for 1 h, and the pH was adjusted to 8.7 and 10.7 using 1.0 M HCl and 0.5 M NaOH solutions. For solution studies with glucose, 0.1 mL of 0.5 M glucose solution was added to DLS samples. Samples were filtered with a 0.45 μm nylon syringe filter, and DLS measurements were recorded at 25° C.

(ii) Polymerization of Boronic Ester Monomers Followed by Subsequent Deprotection to Yield Boronic Acid-Containing Polymers

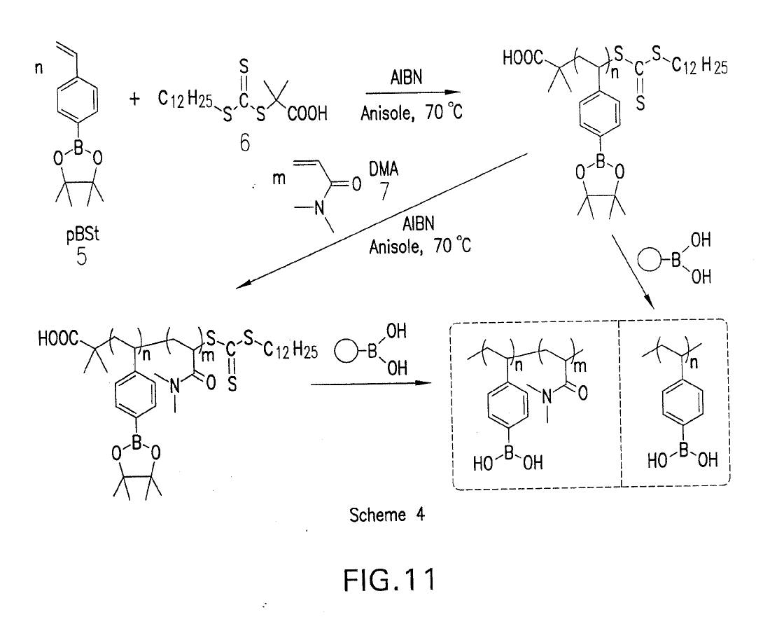

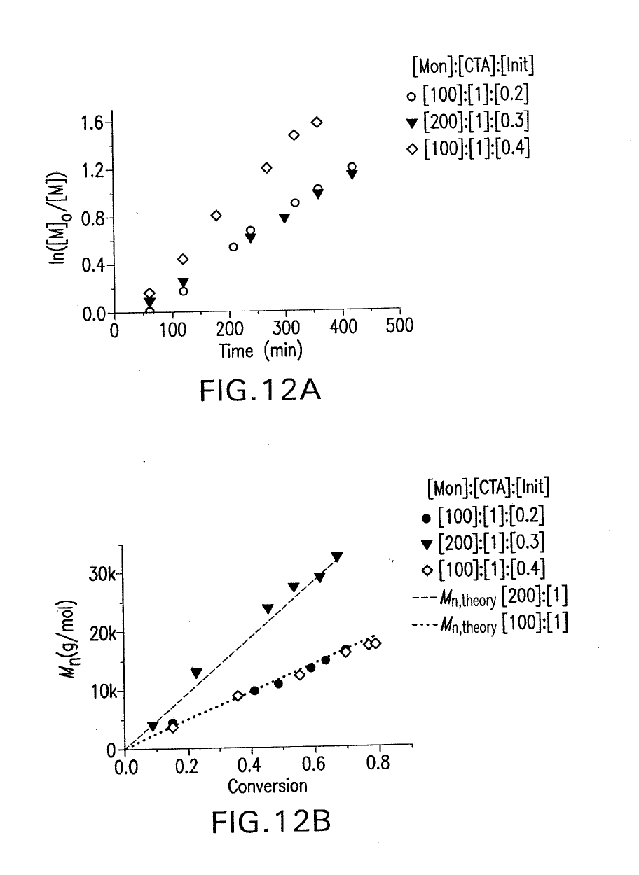

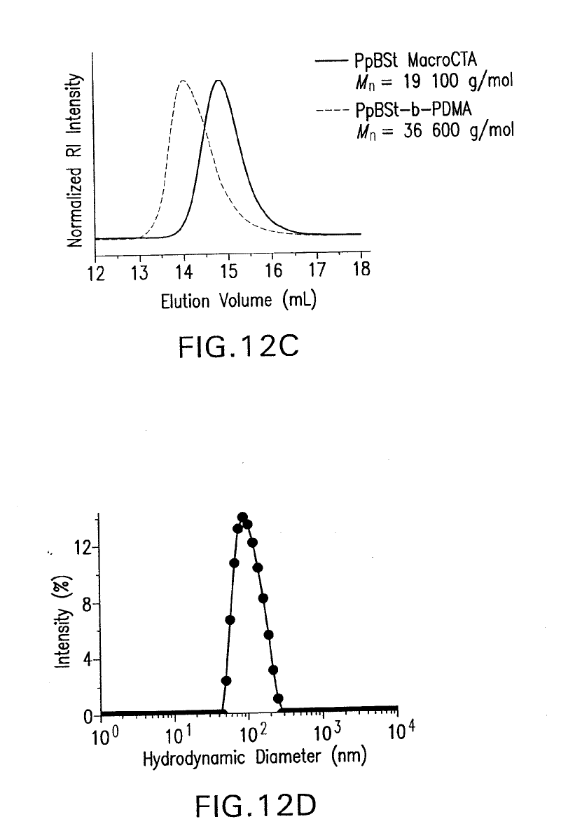

In one case, 4-pinacolatoborylstyrene (pBSt, 5), the pinacol ester of 4-vinylphenylboronic acid, was polymerized by RAFT with 2-dodecylsulfanylthiocarbonylsulfanyl-2-methyl-propionic acid (6) as the chain transfer agent (CTA) and 2,2′-azobisisobutyronitrile (AIBN) as the initiator (FIG. 11, Scheme 4). The polymer was subsequently deprotected to obtain the boronic acid (co)polymers. Agreement between theoretical and experimental molecular weights was excellent (FIG. 12B). Poly(pBSt) (PpBSt) homopolymers were employed as macro chain transfer agents to synthesize block copolymers with DMA (7) (FIG. 11, Scheme 4). Successful chain extension confirmed end group retention, while simultaneously leading to amphiphilic diblock copolymers. Indeed, PpBSt₁₄₅-b-PDMA₂₇₃ (Mₙ=60,800 g/mol, M_{w}/Mₙ=1109) formed micelles of approximately 98 nm in water, as determined by dynamic light scattering (DLS)(FIG. 12D).

Postpolymerization modification of silylated precursors as well as deprotection of stable and conveniently manipulated polymeric pinacol esters has been used to arrive at the polymeric boronic acids of the disclosure. Generally, rather harsh conditions may be necessary to deprotect hindered boronic esters. Transesterification with another boronic acid followed by hydrolysis may be used, but the requirement of an excess of this second free boronic acid significantly complicates purification and separation. Some methods of the present disclosure may overcome this problem by transesterification of the PpBSt units with excess boronic acid immobilized on an insoluble support. Purification was simplified, and the efficiency of pinacol removal was essentially quantitative, as determined via ¹H NMR spectroscopy by the disappearance of the pinacol ester peaks. For the block copolymers with PDMA, deprotection occurred without degradation of the acrylamido units.

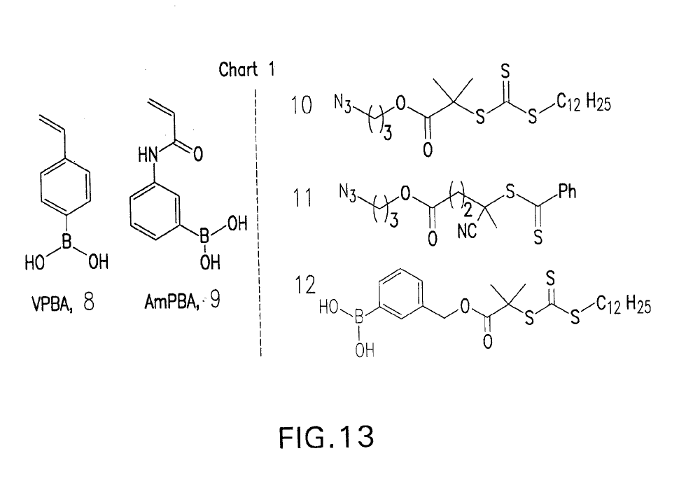

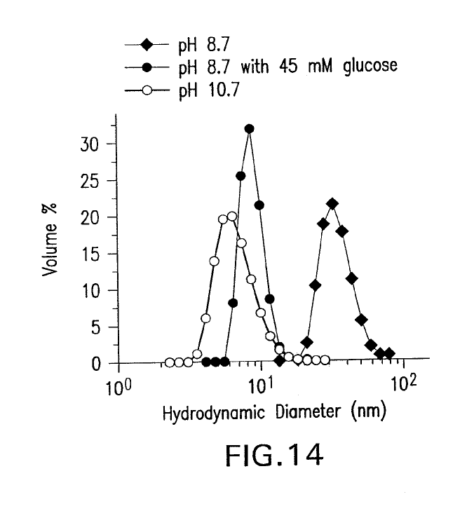

Successful RAFT polymerization of non-protected, free boronic acid monomers is also demonstrated herein. The present disclosure may include free boronic acid monomers being polymerized by any controlled/living method. Well-defined styrenic and acrylamido polymers resulted from the polymerization of 4-vinylphenylboronic acid (VPBA, 8) and 3-acrylamidophenylboronic acid (AmPBA, 9) with CTA 6 (See FIG. 13, Chart 1). Polymerizations were conducted in DMF with 5% water (to prevent crosslinking via boroxine anhydride trimerization). Molecular weight control was excellent. For example, a polymerization of 9 (See FIG. 13, Chart 1), with a theoretical Mₙ=27,600 g/mol resulted in polymer with Mₙ=27,000 g/mol and M_{w}/Mₙ=1.12. Importantly, micelles prepared from PAmPBA-b-PDMA block copolymers successfully dissociated in aqueous media upon the addition of glucose, representing the first example of sugar/diol-responsive block copolymers (FIG. 14). Similarly, these boronic acid polymers successfully complexed with model diols (i.e., pinacol and Alizarin Red) in organic solution.

### Example 2

### End Group Functionalization Via Raft and Azide-Alkyne Click Chemistry

In addition to providing well-defined pendant boronic acid polymers, some embodiments of the disclosure may include polymers with high degrees of end group functionalization. Telechelics may be employed to create larger macromolecular assemblies, and precise knowledge of end group stoichiometry may be extremely useful to ensure well-defined higher order structures. With a Cu^{I} catalyst, Huisgen azide-alkyne cycloaddition results in highly efficient preparation of 1,4-disubstituted 1,2,3-triazole products. The reaction may be conducted under moderate conditions in aqueous or organic media with little or no side products. The versatility of the process led to its inclusion in the class of efficient reactions termed “click chemistry.”

Some embodiments of the present disclosure may include preparation of ω-(meth)acryloyl macromonomers via ATRP and azide-alkyne coupling. This is an efficient and specific means to prepare macromonomers from any monomer polymerizable by ATRP. In some embodiments, a method may be further used to with other radically polymerizable monomer classes. Accordingly, combination of click chemistry and RAFT were used herein to prepare telechelic polymers from monomers not easily controlled by ATRP. Using novel CTAs 10 and 11 (FIG. 13, Chart 1), a variety of functional telechelics were successfully prepared. In addition to postpolymerization modification of (co)polymer chain ends and conjugation to alkyne-labeled proteins and biologically-relevant ligands, low molecular weight azido CTAs were successfully functionalized prior to polymerization.

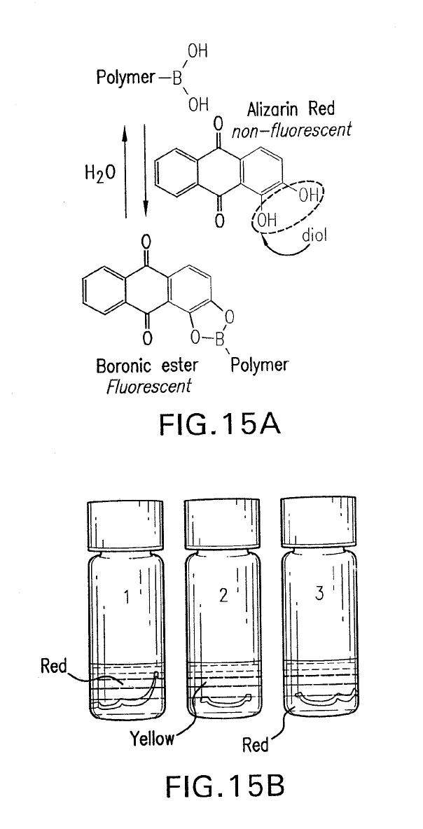

Additionally, boronic acid-containing CTA 12 (FIG. 13, Chart 1) was synthesized, which successfully mediated polymerizations of acrylamido, acrylate, and styrenic monomers, all while retaining the boronic acid end group. Moreover, it has been demonstrated herein that these end groups reversibly bind to model diol compounds, like the catechol dye Alizarin Red (FIG. 15A). FIG. 15A depicts boronic acid-terminal polymer reversibly complexing with a model diol in THF. The reversibility of this complex is shown in FIG. 15B, in which (1) is Alizarin red, (2) is Alizarin Red+PDMA-B(OH)₂, (3) and Alizarin Red+PDMA-B(OH)₂+water (after hydrolysis of the boronic ester).

Examples 1 and 2 above have demonstrated the ability to prepare well-defined boronic acid-containing polymers by RAFT. Controlled polymerization of unprotected boronic acid monomers and simplifying the synthesis of the (co)polymers required has been demonstrated. It has also been demonstrated that the self-assembly behavior of the resulting block copolymers is responsive to diols, exemplified by glucose. By employing a boronic acid CTA, preparation of polymers with boronic acid end groups capable of forming reversible covalent complexes with model diols has been shown. In some embodiments, combining this with the ability to functionalize chain ends by azide-alkyne coupling may provide well-defined polymers with boronic acid or diol end groups. Thus, in some embodiments, the disclosure provides methods to design well-defined polymers with boronic acid or diol end groups.

### Example 3

### Stimuli-Responsive Block Copolymer Assemblies

Stimuli-responsive polymers may undergo marked changes in their physicochemical properties when exposed to external stimuli. In aqueous media, such polymers typically undergo a change in character of functional groups from hydrophilic to hydrophobic, or vice versa. In the unique case of a “smart” block copolymer where one block is hydrophilic and the other stimuli-responsive, the copolymer character may be tuned to be either double-hydrophilic or amphiphilic, depending on the presence or absence of the stimulus. Selective desolvation of the responsive block leads to reversible self-assembly into nanoaggregates such as polymeric micelles, vesicles, or higher order morphologies. Smart block copolymers offer considerable promise in the area of controlled transport and delivery. Polymeric micelles solubilize non-polar species in their hydrophobic cores, while polymeric vesicles can encapsulate water-soluble materials. Upon application of an appropriate stimulus, these nanoaggregates disassemble and release their payload. For example, see FIG. 17.

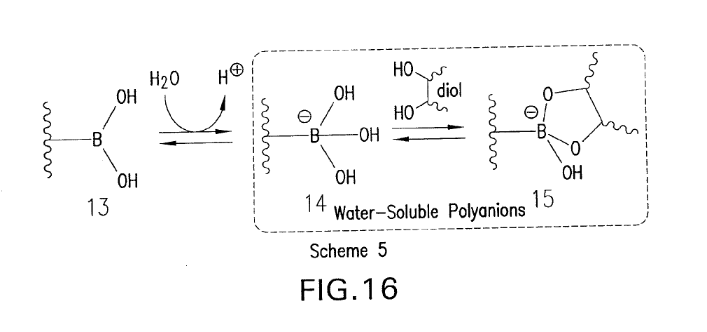

Boronic acids are uniquely stimuli-responsive in that their water solubility is tunable by changes in both pH and solution diol concentration. In aqueous media, boronic acids exist in equilibrium between neutral (hydrophobic/insoluble)(13) and anionic (hydrophilic/soluble)(14) forms (FIG. 16, Scheme 5). Scheme 5 (FIG. 16), depicts aqueous ionization of boronic acids. Complexes between 13 and diols are usually hydrolytically unstable, but 14 readily forms cyclic boronate esters (15) in the presence of 1,2- or 1,3-diols (FIG. 16, Scheme 5). An increase in the concentration of 15 shifts the equilibrium from 13 toward 14, effectively lowering the pKₐ of the acid (FIG. 16, Scheme 5). Thus, complexation adjusts the overall equilibrium from neutral/hydrophobic boronic acid moieties to anionic/hydrophilic boronates. Therefore, the extent of ionization (and water solubility) of boronic acid units increases with diol concentration. Accordingly, in some embodiments, boronic acid block copolymers may be induced to self-assemble at pH<pKₐ, and subsequent diol-dependent solubility may be exploited to trigger micelle and vesicle disassembly. For example, see FIG. 17.

### Example 4

### Methodologies

All references disclosed in U.S. Provisional Patent Application No. 61/086,064 are incorporated herein in material part. Material parts of those references may provide methodologies useful in embodiments of the current disclosure when combined with the teachings of this disclosure.

### Prophetic Example 5

### Block Copolymer Design and Synthesis

A range of diblock copolymers may be prepared from selected hydrophilic monomers and boronic acid monomers of varying pKₐ. Block ratios may be optimized to obtain predetermined morphologies (micelles/vesicles). The resulting solution self-assembly/disassembly may be characterized as a function of molecular architecture, block functionality, copolymer composition, pH, and the concentration of diols. The controlled release of model compounds may be evaluated and optimized.

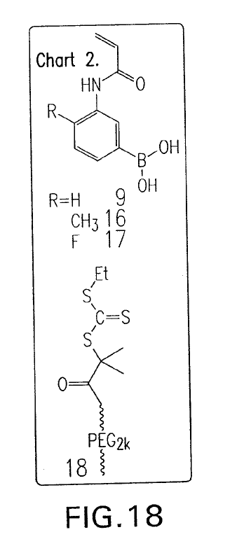

Initially, four boronic acid-containing monomers may be employed: the commercially available VPBA (8) and three (acrylamido)phenylboronic acid monomers (9, 16, 17) (FIG. 18, Chart 2). Due to phenyl ring substitution on these acrylamido monomers, the pKₐ of each boronic acid may vary, thereby determining the pH at which the diol response is most significant. These monomers contain electron rich and electron poor substituents to provide systems with varying pH response ranges. Fluorine substituted 17 (pKₐ≈7.8) (FIG. 18, Chart 2), may be included to provide the best response near physiological pH—an advantage for future therapeutic applications. It has been demonstrated in the Examples above that 9 (FIG. 18, Chart 2), may be polymerized by RAFT, and the other monomers behave similarly. The hydrophilic component of the block copolymers may be prepared from DMA (7), 2-acrylamido-2-methyl-1-propane sulfonic acid (AMPS), or a poly(ethylene glycol)(PEG) RAFT agent (18). These monomers were selected to yield neutral and anionic hydrophilic blocks to observe the effects of electrostatic repulsion and osmotic potential in determining aggregation behavior.

As determined from Examples above, CTA 3 may be employed as the RAFT agent to afford molecular weight control and chain end retention. Boronic acid homopolymers will be prepared from either the free or protected boronic acid monomers (5, 8, 9, 16, 17). These macroCTAs may be used for block copolymerization with the hydrophilic monomers. As described in the sections above, block copolymers with predetermined molecular weight and composition may be prepared according to embodiments of the disclosure. Molecular weights and molecular weight distributions may be determined by size exclusion chromatography (SEC) with refractive index, UV-Vis, light scattering, and viscosity detection. Copolymer compositions may be determined by ¹¹B, ¹H, and ¹³C NMR.

### Prophetic Example 6

### Self-Assembling Copolymers and Characterization Thereof

Provided herein are methods to design and synthesize novel boronic acid-containing block copolymers that reversibly self-assemble in response to biologically-relevant small molecules to form micelles and vesicles with tunable size, morphology, and controlled release potential (FIG. 17). Traditional stimuli employed to induce block copolymer assembly in water include changes in pH, salt concentration, or temperature, representing a rather narrow range of potential triggering mechanisms. The present disclosure provides methods for inducing solution aggregation in response to specific naturally occurring molecules, thereby providing smart polymeric materials with biological relevance.

As demonstrated in the Examples, self assembly of the responsive block copolymers of the disclosure may be accomplished by molecularly dissolving dilute solutions of the block copolymers above the critical micelle concentration (CMC—determined via dynamic light scattering (DLS)) and above the pKₐ of the boronic acid units. Subsequent acid titration (or dialysis against a low pH solution) neutralizes the boronic acid units, rendering the corresponding block insoluble. The gradual transition in solubility during titration/dialysis leads to near-equilibrium solution morphologies.

A range of block copolymers may be characterized to investigate the effect of molecular weight and block length ratio on the resulting aggregate morphology. Near symmetric block lengths may yield micelles, while decreasing hydrophilic segment length may result in rod-like micelles and eventually form vesicles. Systematic block length variation may provide answers to fundamental structure-property questions. For example, effects of block length and character on aggregate morphology, size, and polydispersity may be determined. The CMC may be determined and effect of secondary interactions within the core and corona may be determined. Size and aggregate structure may be addressed through a combination of static/dynamic light scattering (S/DLS), pulsed-gradient spin echo NMR spectroscopy, transmission electron microscopy (TEM), and small-angle neutron scattering. Size characterization over wide ranges of pH, block length, and temperature by high throughput DLS experiments are contemplated.

In some embodiments, naturally occurring diols like glucose, fructose, and adenosine, may trigger solubility transformations in responsive boronic acid-containing blocks, allowing release of therapeutic compositions contained in micelles and vesicles, such as self-assembling micelles or vesicles.

Selected diols may be used to investigate the complexation-induced disassembly, including glucose in a concentration range of 0-100 mM (>20 mM=hyperglycemia) in phosphate buffered saline (PBS). This range has been demonstrated to be effective for changes in boronic acid polymer solubility. Other naturally-occurring 1,2-diols (fructose, sucrose, and adenosine) may be investigated as well. Binding constants for each diol may be determined using the method of Wang, which may facilitate fine-tuning of pKₐ and polymer structure such that complexation is highly specific. Aggregate dissolution may be monitored by DLS with an integrated auto-titrator by observing a reduction in hydrodynamic size as a function of diol concentration.

In most aqueous systems, aggregate dissolution in response to diol concentration may occur with a sharp transition. However, for potential applications in biological environments, diol concentration may not expected to be an “on/off” type of stimulus, as there are continuous basal levels of diols present in the system. Thus, the ability to fine tune the responsive nature of the boronic acid block may be vital. This may be accomplished by copolymerization of the boronic acid monomer with small amounts of hydrophilic or hydrophobic co-monomers. Increased hydrophilicity of the responsive boronic acid block may enhance sensitivity and facilitate diol-induced rupture, while the inclusion of hydrophobic monomer units may provide increased aggregate stability.

Understanding of the assembly and dissolution processes by fundamental controlled release studies may involve the solubilization of hydrophobic or hydrophilic model compounds for micellar and vesicular solutions, respectively. Release of the solubilized contents during equilibrium dialysis in PBS may be monitored as a function of diol concentration. For delivery applications insulin may be encapsulated into vesicles for protein solubilization and delivery. Depending on block copolymer composition and molecular weight, vesicle diameters of ca. 20 nm to several microns may be obtained, therefore vesicle formation in the presence of an appropriate concentration of insulin may allow a significant quantity of protein to be engulfed. After encapsulation and purification, release profiles of insulin may elucidate protein delivery kinetics at glucose concentrations that approximate hyperglycemia.

Employing synthetic techniques developed herein, a range of boron-containing (e.g. boronic acid-containing), block copolymers may be prepared to characterize their solution properties as a function of block functionality, block length, and copolymer composition. Selected monomers with diverse polarity and boronic acid pKₐ may be used to examine their effect on the resulting block copolymer solution behavior. Aggregate disassembly and subsequent controlled release may be triggered by introduction of model diols. The resulting structure-property relationships may provide fundamental understanding of polymer self-assembly and facilitate use and development of these nanomaterials as controlled protein delivery systems.

### Prophetic Example 7

### Compositions for Drug Delivery, Including Treatment of Diabetes Mellitus

A composition may include, in some embodiments, biocompatibile and glucose responsive boronic acid polymeric vesicles or micelles. In some embodiments, the biocompatibile and glucose responsive boronic acid polymeric vesicles or micelles of the disclosure may further include a therapeutic agent. The therapeutic agent may be any agent described in the specification or known in the art including peptides, polypeptides, proteins, hormones, steroids, nucleic acids, chemical drugs, pharmaceuticals, and may further incorporate additional agents such as buffers, co-enzymes, metallic components, ions or any other molecule that the therapeutic agent may use to have enhanced or optimum biological function and be stable. For example, insulin may be complexed with Zinc.

In contrast to insoluble macroscopic gels that are sometimes used for insulin delivery, the insulin-loaded polymeric vesicles/micelles of the disclosure may be injectable or orally administrable to a patient in need thereof. They may be designed to reside in the bloodstream for extended periods.

High molecular weight and chain entanglement of macromolecules in the polymeric vesicle membrane may significantly limit permeability to the therapeutic compound. Solubilized contents may be entrapped until the bilayer or other micelle of vesicle wall is disrupted by increased glucose concentration in the bloodstream. In light of the present disclosure, as will be appreciated by one of skill in the art, several other biomedical applications are contemplated and the disclosure is not limited to the treatment of diabetes.

Glucose-responsive polymeric vesicles of the disclosure may be analyzed for their ability as feedback-controlled insulin delivery agents for the treatment of diabetes mellitus. Block copolymers with water solubility dependent on the concentration of glucose in the surrounding medium may be used to construct insulin-loaded vesicles. The vesicles may be designed to rupture under hyperglycemic conditions to release their encapsulated insulin. This controlled release, deliver-as-needed treatment of diabetes is contemplated to lead to increased patient compliance by reducing the number of required injections for blood sugar maintenance. Glucose-responsive polymeric vesicles may be developed by performing one or more of the following tests.

1. Prepare a range of diblock copolymers from selected hydrophilic monomers and boronic acid monomers of varying pKₐ. To enable solution self-assembly into larger supramolecular structures capable of encapsulating and releasing insulin, it may be desirable and/or vital to prepare copolymers containing a responsive (boronic acid) block and a hydrophilic, non-toxic, non-immunogenic (PEG) block. The responsive nature of the insulin-loaded assemblies may arise from binding between the boronic acid moieties and glucose in the surrounding medium and is expected to be most efficient when the pKₐ of the boronic acid groups is near physiological pH.

2. Optimize block ratios to obtain predetermined morphologies (micelles/vesicles). The solution morphology of the self-assembled polymeric aggregates may be directly dependent on the block copolymer molecular weight, composition, and functionality. Because insulin delivery is based on the responsive mechanism, fundamental structure-property relationships to predict solution morphology that may result from a given set of block copolymer characteristics may be determined.

3. Characterize solution self-assembly/disassembly as a function of copolymer concentration, molecular architecture, copolymer composition, pH, and the concentration of diols.

4. Evaluate the controlled release of model compounds. The polymeric vesicles may have a critical aggregation concentration (CAC). The CAC is the floor concentration below which the aggregates are no longer thermodynamically stable and dissociation to individual polymer chains may occur. However, the polymeric aggregates do not immediately dissociate when diluted by injection into the bloodstream because of their remarkably low CAC (10⁻⁶-10-⁷M), which is nearly 1000 times lower than that of low molecular weight surfactants. The vesicles may no longer be thermodynamically stable below this critical concentration, the kinetics of aggregate dissociation are extremely slow as a result of the high molecular weight and entangled nature of the block copolymers composing the vesicular bilayer. Therefore, the vesicles are expected to demonstrate prolonged stability. To evaluate biostability for each sample, polymeric vesicles will be incubated with isolated Kupffer cells in vitro.

5. Prepare well-defined, controlled molecular weight block copolymers from boronic acid-containing monomers and poly(ethylene glycol) (PEG).

6. Evaluate the structure-property relationships between the boronic acid-containing block copolymers and their ability to induce specific self-assembly into polymeric vesicles.

Although only exemplary embodiments of the disclosure are specifically described above, it will be appreciated that modifications and variations of these examples are possible without departing from the spirit and intended scope of the invention.

1. A composition comprising:
a polymer comprising at least one monomeric boron moiety wherein the boron moiety is incorporated pendantly or terminally, wherein said polymer is operable to self-assemble into a micelle or vesicle and to disassemble from the micelle or vesicle in response to an organic stimulus; and
a therapeutic agent operable to be released from the micelle or vesicle in response to the organic stimulus.

2. The composition of claim 1, wherein the boron moiety is a boronic acid.

3. The composition of claim 1, wherein the boron moiety is a boronic ester.

4. The composition of claim 1, wherein the polymer further comprises a block derived entirely or partially from an acrylamide.

5. The composition of claim 4, wherein the acrylamide is selected from a group consisting of poly(N-isopropylacrylamide), polyacrylamide, poly(hydroxymethylacrylamide, or any combination thereof.

6. The composition of claim 1, wherein the polymer further comprises a block derived entirely or partially from a methacrylamide.

7. The composition of claim 1, wherein the polymer further comprises a block derived entirely or partially from an acrylate.

8. The composition of claim 1, wherein the polymer further comprises a block derived entirely or partially from a methacrylate.

9. The composition of claim 1, wherein the polymer further comprises a block derived entirely or partially from a vinyl monomer.

10. The composition of claim 1, wherein the polymer further comprises polyethylene glycol.

11. The composition of claim 1, where in the polymer comprises a block copolymer comprising monomer units of a boron moiety containing at least one pendant boronic acid moiety per repeat unit.

12. The composition of claim 1, wherein the organic stimulus comprises glucose.

13. The composition of claim 1, wherein the therapeutic agent comprises insulin.

14. A method for preparing a polymer comprising at least one monomeric boron moiety wherein the boron moiety is incorporated pendantly or terminally comprising:
direct polymerization or copolymerization of boronic acid-containing or boronic ester-containing monomers; and
deprotection to boronic acid moieties or boronic ester moieties.

15. A method for the administration of a therapeutic agent comprising:
administering to a mammal a block copolymer micelle or vesicle comprising:
at least one monomeric boron moiety wherein the boron moiety is incorporated pendantly or terminally; and
a pharmaceutical formulation of the therapeutic agent;
wherein the release of the therapeutic agent in the mammal comprises dissolution of the block copolymer micelle or vesicle.

16. The method of claim 15, wherein the dissolution of the block polymer is triggered by an increase in the local or global concentration in the mammal of a 1,2-diol or a 1,3-diol.

17. The method of claim 15, wherein the 1,2-diol or a 1,3-diol is a saccharide selected from the group containing glucose, fructose, and sucrose.

18. The method of claim 15, wherein the therapeutic agent release is induced by dissolution of the block polymer micelles or vesicles triggered by an increase in the local or global concentration of a 1,2-diol or a 1,3-diol.

19. The method of claim 15, wherein the therapeutic agent is insulin.

20. The method of claim 15, wherein the administration comprises oral administration, sublingual administration, parenteral administration, topical administration, administration to eye or mucosal membranes.
